(19) United States
(12) Patent Application Publication
(10) Pub. No.: US 2003/0027196 A1
(43) Pub. Date: Feb. 6, 2003

(54) Magnesium precipitate methods for magnesium dependent enzymes

(76) Inventors: Wayne M. Barnes, University City, MO (US); Katherine R. Rowlyk, Kentfield, CA (US)

Correspondence Address: G. Harley Blosser, Sonnenschein Nath & Rosenthal, P.O. Box #061080, Wacker Drive Station, Sears Tower, Chicago, IL, 60606-1080, US

(21) Appl. No.: 10/211,867

(22) Filed: Aug. 2, 2002

Related U.S. Application Data
(60) Provisional application No. 60/309,646, filed on Aug. 2, 2001.

(51) Int. Cl. C12Q 1/68; C12P 19/34

(52) U.S. Cl. 435/6; 435/91.2

(57) ABSTRACT

The present invention provides methods of performing enzymatic reactions which require the use of magnesium dependent enzymes, including restriction endonucleases, ligases, and reverse transcriptases. The method is based on sequestration of magnesium ions in the form of a precipitate which renders a magnesium dependent enzyme inactive until the appropriate time in the reaction when a certain temperature is reached and the magnesium ions are released from the precipitate. Also provided are kits comprising reagents and instructions for DNA digestion and ligation, and for reverse transcription of RNA into cDNA. Furthermore, the kits and reagents of the present invention can be utilized in other reactions requiring magnesium dependent enzymes.

### CROSS-REFERENCE TO RELATED APPLICATIONS

This application claims the benefit and priority of co-pending U.S. provisional application serial No. 60/309,646, filed Aug. 2, 2001 as well as co-pending U.S. non-provisional application serial No. 10/091,784 filed Mar. 6, 2002 and its parent, U.S. non-provisional application serial No. 09/920,872, filed Aug. 2, 2001 and issued Jun. 11, 2002 as U.S. Pat. No. 6,403,341, all the specifications of which are included herein by reference as if restated here in full.

### FIELD OF THE INVENTION

The present invention is directed to a novel method of performing enzymatic reactions involving magnesium dependent enzymes which are active at temperatures above 30° C., such as ligases, restriction endonucleases, and reverse transcriptases. Furthermore, the present invention relates to achieving a greater specificity of the above-mentioned reactions. Also provided in the present invention are reagents and kits for performing enzymatic reactions using a magnesium precipitate.

### BACKGROUND OF THE INVENTION

Recombinant DNA technology has become widely used in recent years, has contributed to major scientific breakthroughs and relies heavily on the use of enzymes such as restriction endonucleases, ligases, and reverse transcriptases.

Restriction endonucleases naturally occur in bacteria, and isolated and purified forms of such nucleases can be used to “cut” DNA molecules at precise locations. These enzymes function by first recognizing and binding to a particular double-stranded sequence (“recognition sequence”) within the DNA molecule. Once bound, they cleave the DNA molecule either within or to one side of the recognition sequence to which they are bound. The majority of restriction endonucleases recognize sequences that are four to six nucleotides in length; however, a small number of endonucleases can cleave sequences that are seven to eight nucleotides in length. The target DNA must be double-stranded for the restriction enzymes to bind and cleave. Apparent cleavage of single-stranded DNA is actually due to the formation of double-stranded regions by intrastrand folding at ambient to warm temperatures (20° to 30° C.).

The temperature at which restriction enzymes are active varies; however, many enzymes prefer temperatures above the ambient temperature. For example, 98% of enzymes available from New England BioLabs have optimum activities above 30° C. Some 5% of the restriction enzymes are active at temperatures above 55° C. All restriction endonucleases require magnesium ions for activity.

The second group of enzymes which are important in recombinant DNA technology are ligases. These enzymes are responsible for joining or ligating DNA molecules through a reaction involving the 3′-hydroxy and 5′-phosphate termini. In vivo, one of the functions of DNA ligases involves fixing DNA damage which the ligase accomplishes by utilizing a molecule of ATP or NAD⁺ to activate the 5′ end at the nick in the DNA prior to forming a new bond. With regard to recombinant DNA molecules, the process is the same with the exception that the DNA ligase “seals” cohesive ends produced by restriction endonucleases instead of the nicks in the DNA. In case of blunt ends, the ligation process is less efficient since base-pairing does not occur between the termini. Therefore, ligation reactions with blunt ends require higher concentrations of DNA and ligase in the reaction mixtures. See U.S. Pat. No. 6,143,527.

In addition to ligation of recombinant DNA molecules, an important in vitro use of ligase is in ligase chain reaction (LCR) which is an alternative to PCR in target nucleic acid amplification. LCR utilizes thermostable ligases, which are active at higher temperatures than regular ligases. For instance, Taq ligase, isolated from Thermus aquaticus, functions optimally at temperatures between 45° C. and 65° C. In LCR reactions, repeated cycles of hybridization and ligation of primary and secondary probes result in amplification of the target sequence. See U.S. Pat. No. 5,427,930. LCRs have been utilized in DNA diagnostics such as genetic disease detection since they can detect single-base mismatches in DNA targets, thereby indicating the mutated or disease-causing alleles. See Barany, Proc. Natl. Acad. Sci. USA, Vol. 88, pp. 189-193, January 1991.

One of the problems of achieving specificity in LCR is the ligation of the probe primers when they are non-specifically annealed to non-target DNA during reaction setup. This can cause a seed of competing signal that confounds the specific detection and quantization of the desired specific sequence(s).

Reverse transcriptases (RT) were first recognized as components of retroviruses whose genetic material consists of single-stranded RNA. These viruses use RTs to synthesize a complementary DNA strand (cDNA) using viral RNA as a template, which is followed by the synthesis of double stranded DNA and subsequent integration into the host genome. See U.S. Pat. No. 5,998,195. At present, reverse transcriptases are frequently used in molecular biology because of their ability to synthesize complementary DNA from almost any RNA template. Thus, reverse transcriptase is commonly used to make nucleic acids for hybridization probes and to convert single-stranded RNA into a single-stranded cDNA, which can further be converted into a double-stranded DNA for subsequent cloning and expression by techniques such as PCR.

Reverse transcriptases have been used as a component of transcription-based amplification systems that can amplify RNA and DNA target sequences up to 1 trillion fold. See e.g., PCT Patent Application WO 89/01050 and European Patent Application EP 0329822. Reverse transcriptases are also included in RT-PCR reactions wherein an initial step involves making a cDNA copy of the RNA target, which is then amplified by PCR. See U.S. Pat. No. 5,998,195. Similarly to PCR reactions, RT-PCR reactions are very sensitive to a variety of factors such as magnesium concentration and pH, and can result in production of nonspecific bands if RT can non-specifically initiate the synthesis of cDNA.

One method of improving the specificity of PCR reactions is to keep one critical component, usually magnesium, absent from the reaction until a temperature is reached that ensures specificity of primer annealing. The withholding of a key reagent is commonly utilized in PCR reactions and can be done manually by adding magnesium only after the desired temperature is reached. This method is often referred to as “manual hot start”.

Accordingly, a method which would withhold a critical component, such as magnesium from magnesium dependent restriction endonucleases, ligases, and reverse transcriptases which are active above 30° C. would be desirable to improve the specificity of such enzymatic reactions. As such, a need exists to provide novel or modified methods of performing enzymatic reactions involving magnesium dependent enzymes that would allow for improved precision and specificity of the reactions.

### SUMMARY OF THE INVENTION

Among the several aspects of the invention, therefore, may be noted the provision of novel processes for performing enzymatic reactions which require the use of a magnesium dependent enzyme such as a restriction enzyme, ligase or reverse transcriptase. These magnesium dependent enzymes are typically utilized in reactions which occur at temperatures above 30° C. Briefly, the present invention is directed to processes of cleaving DNA using restriction endonucleases, ligating DNA using DNA ligases, and transcribing RNA into cDNA using reverse transcriptases. Accordingly, the present invention provides reagents and kits which can be used to perform said reactions.

In particular, the processes of the invention comprise sequestering magnesium ions in a precipitate thereby rendering the magnesium dependent enzyme such as a restriction endonuclease, a ligase, or a reverse transcriptase inactive until the magnesium ions are released. In one aspect, the processes of the present invention utilize a reagent which comprises a precipitate containing magnesium. Alternatively, the reagent comprises a source of magnesium ions with a source of phosphate ions which can be used to form a precipitate combining the source of magnesium ions and the source of phosphate ions at a temperature of below 34° C. These reagents are utilized in enzymatic reactions including cleaving of DNA, reverse transcribing DNA and ligating DNA molecules, which occur at temperatures above 30° C. in order to improve the specificity of such reactions.

A further aspect of the present invention is to provide kits useful for reactions involving magnesium dependent enzymes. These enzymes include restriction endonucleases, ligases and reverse transcriptases. In one embodiment, kits of the present invention comprise a container containing a source of magnesium ions and a container containing a source of phosphate ions which form a precipitate containing magnesium when combined at temperatures below the temperature at which enzymatic manipulation occurs, such as below 34° C., and instructions for performing said reactions. In another embodiment, the kits comprise a container containing a reagent comprising a precipitate containing magnesium and instructions for using the precipitate containing magnesium. Preferably, other reagents necessary for the above-mentioned reactions are included in the kits of the present invention.

Other aspects and features will be in part apparent and in part pointed out hereinafter.

### DETAILED DESCRIPTION

All publications, patents, patent applications or other references cited in this application are herein incorporated by reference in their entirety as if each individual publication, patent, patent application or reference are specifically and individually indicated to be incorporated by reference.

Abbreviations and Definitions

The listed abbreviations and terms, as used herein, are defined as follows:

“Reverse transcriptase” is defined herein as an RNA-directed DNA polymerase or as a DNA polymerase exhibiting reverse transcriptase ability.

Taq is the abbreviation for Thermus aquaticus.

Tth is the abbreviation for Thermus thermophilus.

rTth is the abbreviation for recombinant thermostable polymerase obtained from Thermus thermophilus that possesses reverse transcriptase and Taq-like DNA polymerase activities.

“RT reaction” or “reverse transcriptase reaction” are herein used interchangeably to indicate a reverse transcriptase catalyzed reaction, wherein a target RNA sequence is transcribed into cDNA.

“RT-PCR” or “reverse transcriptase polymerase chain reaction” is a reaction in which replicate DNA copies are made of a target RNA sequence using one or more primers, and catalysts of polymerization, such as reverse transcriptase and DNA polymerase, and particularly thermostable forms of these enzymes. Generally, a target RNA sequence is first reverse transcribed into cDNA by the action of reverse transcriptase. Subsequently, PCR is performed, wherein the cDNA can be amplified many times depending on the number of PCR cycles. For instance, twenty amplification cycles can yielded up to a million-fold amplification of the target DNA sequence. Methods for PCR amplification are taught in U.S. Pat. Nos. 4,683,195 and 4,683,202. For RT-PCR, see e.g., U.S. Pat. Nos. 5,130,238 and 5,693,517.

“Single restriction enzyme digest” or “restriction enzyme reaction” are used interchangeably herein to refer to reactions catalyzed by a single restriction enzyme that cleaves target DNA at specific sites either within or at the ends of DNA molecule(s).

“Multiple restriction enzyme digest” or “multiple restriction enzyme reaction” are used interchangeably herein to indicate reactions catalyzed by multiple restriction enzymes that cleave target DNA molecule at their cognate sites either within or at the ends of the DNA molecules.

“Ligase reaction” as used herein refers to a reaction catalyzed by a ligase, which results in ligation or joining of target nucleic acid sequences through formation of phosphodiester bonds between 5′ and 3′ termini of the target nucleic acids.

“Specificity” in RT-PCR reaction refers to the generation of a single, “specific”, RT-PCR product with the size and sequence predicted from the sequences of the primers and the genomic or transcribed region of nucleic acid to which the primers were designed to anneal in a base-complementary manner. “Specificity” in a single or a multiple restriction enzyme digest refers to the ability of restriction enzyme(s) to only cleave DNA at their cognate recognition sequences in double-stranded form without cleaving any other similar, non-specific or single-stranded DNA sequences. “Specificity” in a ligase reaction refers to the ability of the ligase to specifically join two or more DNA sequences only when their 5′ and 3′ ends being joined are fully double-stranded and base-paired for at least few bases or for the length of the oligonucleotide substrate probes.

The procedures disclosed herein which involve the molecular manipulation of nucleic acids are known to those skilled in the art. See generally Frederick M. Ausubel et al. (1995), “Short Protocols in Molecular biology”, John Wiley and Sons, and Joseph Sambrook et al. (1989), “Molecular Cloning, A Laboratory Manual”, second ed., Cold Springs Harbor Laboratory Press, which are both incorporated by reference.

Accordingly, the present invention provides processes and kits for performing reactions requiring magnesium dependent enzymes. Preferably, these enzymes comprise ligases, restriction endonucleases, and reverse transcriptases. The enzymes utilized in these processes are magnesium dependent and the enzymatic reactions in which the enzymes are utilized generally occur at temperatures above 30° C. The processes and kits utilize the step of sequestering magnesium ions, thereby rendering a magnesium dependent enzyme inactive until the magnesium ions are released from the precipitate into the reaction mixture.

The magnesium precipitate method of the present invention is achieved by forming a precipitate comprising magnesium ions which sequesters the magnesium ions from other reaction reagents and preferably, prevents significant magnesium dependent enzyme activity due to the lack of magnesium ions in the reaction mixture. The magnesium ions utilized in the present invention are available from different sources. Preferably, the sources of magnesium ions include but are not limited to magnesium chloride, magnesium hydroxide, magnesium carbonate and magnesium sulfate. In a preferred embodiment, the source of magnesium ions is magnesium chloride.

Many sources of phosphate ions are available in the art. Preferably, the sources of phosphate ions include but are not limited to phosphoric acid (H₃PO₄), potassium phosphate (K₂HPO₄), and ammonium phosphate ((NH₄)₂HPO₄). In a preferred embodiment, the source of phosphate ions is ammonium phosphate or phosphoric acid and more preferably, the source of phosphate ions utilized is phosphoric acid.

Many buffers used in reactions utilizing restriction enzymes, ligases, or reverse transcriptases contain magnesium. As such, the processes of the present invention may utilize buffers which contain the source of magnesium ions for the formation of the magnesium precipitate. In this embodiment, the magnesium precipitate method is achieved by adding a source of phosphate ions to a buffer containing magnesium ions to form a precipitate containing magnesium. Preferably, this buffer containing magnesium ions is at higher concentration i.e., contains less water, than the concentration of the reaction mixture at which the enzymatic process occurs.

In a preferred embodiment, the source of phosphate ions is contained in a solution which is buffered to a pH above 7. Solutions or buffers used for performing reactions with magnesium dependent enzymes vary depending on the enzyme used. For ligase reactions, the buffer often comprises Tris (for pH stabilization), a source of magnesium ions, a reducing agent, preferably dithiothreitol (DTT), and bovine serum albumin (BSA) or a surfactant for preventing aggregation of enzyme, a salt, preferably potassium acetate. If Taq ligase the ligase utilized in the reaction, then the buffer will also contain NAD+ co-factor. For RT-PCR reactions, the buffer commonly comprises Tris, a source of magnesium ions, a reducing agent such as DTT, and a salt such as potassium chloride. Buffers for restriction enzymes vary in specific content but commonly contain Tris, a salt, usually sodium chloride or potassium acetate, and a reducing agent such as DTT. The required concentrations of these buffer components will vary depending on the magnesium dependent enzyme. Such concentrations would be easily determined by one skilled in the art.

Alternatively, buffers may be utilized in the enzymatic process which are not pre-formulated with a source of magnesium or a source of phosphate ions. In this case, either the source of magnesium ions or the source of phosphate ions can first be mixed with the buffer and incubated with either the source of phosphate ions or the source of magnesium ions, respectively, to form a precipitate containing magnesium. This is another way of achieving all the benefits of magnesium precipitate method for magnesium dependent enzymes.

The precipitate is formed by combining a source of magnesium ions and a source of phosphate ions for at least 3 minutes at a temperature below 34° C., preferably ranging from 4° to 30° C. and preferably, at 4° C. The incubation of phosphoric acid with magnesium ions for approximately 3 minutes at a low temperature produces an insoluble precipitate containing magnesium and phosphate. Preferably, the source of magnesium ions and the source of phosphate ions are incubated at a temperature of at least 4° C. In another preferred embodiment, the source of magnesium ions and the source of phosphate ions are incubated at a temperature of at least 25° C. In yet another preferred embodiment, the source of magnesium ions and the source of phosphate ions are incubated at a temperature of 0° to 30° C. The source of magnesium and the source of phosphate are incubated for at least three minutes to form the precipitate containing magnesium. Preferably, the source of magnesium and the source of phosphate are incubated for at least 5 minutes and more preferably, for at least 10 minutes.

In a preferred embodiment, the source of phosphate ions is incubated with a source of magnesium ions in a concentration at or above appropriate for a particular enzyme and for a particular enzymatic reaction, at a temperature of 4° to 30° C. for at least 5 minutes, more preferably 15 minutes, to form a precipitate containing magnesium.

Once the precipitate is formed, the additional reagents appropriate for the enzymatic reaction being performed are added. In case of single or multiple restriction enzyme digests, the commonly added reagents include sterile nuclease-free water, a target DNA sample, and restriction enzyme(s). For ligase reactions, the additional reagents to be added are target DNA molecule(s), and a particular ligase, preferably Taq ligase. If Taq ligase is utilized, then co-factor NAD+ is also added to the reaction mixture. RT reactions would require addition of a target RNA sequence, at least one primer, deoxyribonucleosides, and a reverse transcriptase. Hot start RT-PCR reactions require the addition of a target RNA sequence, at least one primer, deoxyribonucleosides, and an enzyme or mixture of enzymes possessing both RT and DNA polymerase activities (such as rTth) .

After the precipitate is combined with other reaction reagents to form a reaction mixture, the magnesium is released from the precipitate and into the reaction mixture. The release of the magnesium ions into the reaction mixtures results in making the magnesium available to the enzyme and consequentially, activating the magnesium dependent enzyme for the desired enzymatic process. The ability of the precipitate to sequester magnesium until the appropriate conditions are achieved to release the magnesium results in increased specificity of the reaction and/or simultaneous start of a number of reactions. Preferably, the mixture containing the precipitate and reaction reagents is heated to standard temperatures required for the reaction being performed so that the magnesium is released from the precipitate at a higher temperature than the temperature at which nonspecific DNA ligation, digestion or RNA reverse transcription occur, and more preferably, the magnesium ions are released by heating the reaction mixture to a temperature above 30° C. In this way, the magnesium precipitate method provides an improved specificity for reactions involving magnesium dependent enzymes. The temperature at which the precipitate dissolves is achieved during the standard reaction temperatures, thereby eliminating any extra steps and need for additional reagents.

Besides a greater precision and specificity, the magnesium precipitate method possesses other beneficial attributes such as the ease of manipulation, the little extra time necessary to perform it, and the inexpensive reagents required. The processes of the present invention are not only useful in reactions specified above, but can also be applied in any reaction that requires use of a magnesium dependent enzyme.

A further aspect of the present invention is to provide kits useful for reactions involving magnesium dependent enzymes. These enzymes include restriction endonucleases, ligases and reverse transcriptases. In one embodiment, kits of the present invention comprise a container containing a source of magnesium ions and a container containing a source of phosphate ions which form a precipitate containing magnesium when combined at temperatures of below 34° C. and instructions for performing said reactions. In another embodiment, the kits comprise a container containing a reagent comprising a precipitate containing magnesium and instructions for using the precipitate containing magnesium. Preferably, other reagents necessary for the above-mentioned reactions are included in the kits of the present invention.

It is to be understood that the present invention has been described in detail by way of illustration and example in order to acquaint others skilled in the art with the invention, its principles, and its practical application. Further, the specific embodiments of the present invention as set forth are not intended as being exhaustive or limiting of the invention, and that many alternatives, modifications, and variations will be apparent to those skilled in the art in light of the foregoing examples and detailed description. Accordingly, this invention is intended to embrace all such alternatives, modifications, and variations that fall within the spirit and scope of the following claims. While some of the examples and descriptions above include some conclusions about the way the invention may function, the inventors do not intend to be bound by those conclusions and functions, but puts them forth only as possible explanations.

We claim:

1. A method of molecular manipulation of a nucleic acid sequence with an enzyme, said method comprising:
a. forming or obtaining a reagent comprising a source of magnesium ions and a source of phosphate ions, wherein the source of magnesium ions and the source of phosphate ions form a precipitate at a temperature below a temperature at which specific enzymatic manipulation occurs;
b. making a mixture comprising a precipitate of the reagent of step (a), a magnesium dependant enzyme and the nucleic acid sequence;
c. releasing the magnesium ions into the mixture thereby activating said enzyme; and
d. allowing the enzyme to catalyze the manipulation of said nucleic acid.

2. A method of cleaving a nucleic acid sequence with a restriction endonuclease, said method comprising:
a. forming or obtaining a reagent comprising a source of magnesium ions and a source of phosphate ions, wherein the source of magnesium ions and the source of phosphate ions form a precipitate at a temperature below a temperature at which specific cleaving occurs;
b. making a mixture comprising a precipitate of the reagent of step (a), a restriction endonuclease and the nucleic acid sequence comprising a restriction site for said restriction endonuclease;
c. releasing the magnesium ions into the mixture thereby activating the restriction endonuclease;
d. allowing the restriction endonuclease to recognize and bind to the recognition sequence of the nucleic acid sequence; and
e. cleaving said nucleic acid.

3. A method for reverse transcribing an RNA template, said method comprising:
a. forming or obtaining a reagent comprising a source of magnesium ions and a source of phosphate ions, wherein the source of magnesium ions and the source of phosphate ions form a precipitate at a temperature below a temperature at which specific reverse transcription occurs;
b. making a mixture comprising a precipitate of the reagent of step (a), said RNA template, an oligonucleotide primer, which primer is sufficiently complementary to said RNA template to hybridize therewith, and reverse transcriptase in the presence of all four deoxyribonucleoside triphosphates;
c. releasing the magnesium ions into the mixture thereby activating the reverse transcriptase; and
d. allowing said primer to hybridize to said RNA template and said reverse transcriptase to catalyze the polymerization of said deoxyribonucleoside triphosphates to provide cDNA complementary to said RNA template.

4. A method of ligating nucleic acid sequences with a thermostable DNA ligase, said method comprising:
a. forming or obtaining a reagent comprising a source of magnesium ions and a source of phosphate ions, wherein the source of magnesium ions and the source of phosphate ions form a precipitate at a temperature below a temperature at which specific ligation occurs;
b. making a mixture comprising a precipitate of the reagent of step (a), the DNA ligase and the nucleic acid sequences;
c. releasing the magnesium ions into the mixture thereby activating the thermostable DNA ligase; and
d. allowing the thermostable DNA ligase to ligate nucleic acid sequences.

5. The method of claim 1 wherein the source of magnesium ions is selected from the group consisting of magnesium chloride, magnesium hydroxide, magnesium carbonate and magnesium sulfate.

6. The method of claim 2 wherein the source of magnesium ions is selected from the group consisting of magnesium chloride, magnesium hydroxide, magnesium carbonate and magnesium sulfate.

7. The method of claim 3 wherein the source of magnesium ions is selected from the group consisting of magnesium chloride, magnesium hydroxide, magnesium carbonate and magnesium sulfate.

8. The method of claim 4 wherein the source of magnesium ions is selected from the group consisting of magnesium chloride, magnesium hydroxide, magnesium carbonate and magnesium sulfate.

9. The method of claim 1 wherein the source of phosphate ions is selected from the group consisting of phosphoric acid, potassium phosphate, and ammonium phosphate.

10. The method of claim 2 wherein the source of phosphate ions is selected from the group consisting of phosphoric acid, potassium phosphate, and ammonium phosphate.

11. The method of claim 3 wherein the source of phosphate ions is selected from the group consisting of phosphoric acid, potassium phosphate, and ammonium phosphate.

12. The method of claim 4 wherein the source of phosphate ions is selected from the group consisting of phosphoric acid, potassium phosphate, and ammonium phosphate.

13. The method of claim 1 wherein the source of magnesium ions is magnesium chloride and the source of phosphate ions is phosphoric acid.

14. The method of claim 2 wherein the source of magnesium ions is magnesium chloride and the source of phosphate ions is phosphoric acid.

15. The method of claim 3 wherein the source of magnesium ions is magnesium chloride and the source of phosphate ions is phosphoric acid.

16. The method of claim 4 wherein the source of magnesium ions is magnesium chloride and the source of phosphate ions is phosphoric acid.

17. The method of claim 1 wherein releasing the magnesium ions into the mixture comprises heating reagents to a temperature standard for the enzyme manipulation.

18. The method of claim 2 wherein releasing the magnesium ions into the mixture comprises heating reagents to a temperature standard for cleaving a nucleic acid sequence.

19. The method of claim 3 wherein releasing the magnesium ions into the mixture comprises heating reagents to a temperature standard for reverse transcribing an RNA template.

20. The method of claim 4 wherein releasing the magnesium ions into the mixture comprises heating reagents to a temperature standard for the ligating of the nucleic acid with a thermostable DNA ligase.

21. The method of claim 1 wherein the source of magnesium ions and the source of phosphate ions form a precipitate at a temperature of below 34° C.

22. The method of claim 2 wherein the source of magnesium ions and the source of phosphate ions form a precipitate at a temperature of below 34° C.

23. The method of claim 3 wherein the source of magnesium ions and the source of phosphate ions form a precipitate at a temperature of below 34° C.

24. The method of claim 4 wherein the source of magnesium ions and the source of phosphate ions form a precipitate at a temperature of below 34° C.

25. The method of claim 21 wherein the releasing of the magnesium ions comprises heating the reagents to a temperature of above 30° C.

26. The method of claim 22 wherein the releasing of the magnesium ions comprises heating the reagents to a temperature of above 30° C.

27. The method of claim 23 wherein the releasing of the magnesium ions comprises heating the reagents to a temperature of above 30° C.

28. The method of claim 24 wherein the releasing of the magnesium ions comprises heating the reagents to a temperature of above 30° C.

29. A kit for molecular manipulation of a nucleic acid sequence with an enzyme, said kit comprising:
a. a container containing a source of magnesium ions;
b. a container containing a source of phosphate ions, wherein said source of magnesium ions and said source of phosphate ions form a precipitate containing magnesium when combined at temperatures below a temperature at which specific molecular manipulation occurs; and
c. instructions for performing said molecular manipulations.

30. A kit for cleaving a nucleic acid sequence with an enzyme, said kit comprising:
a. a container containing a source of magnesium ions;
b. a container containing a source of phosphate ions, wherein said source of magnesium ions and said source of phosphate ions form a precipitate containing magnesium when combined at temperatures below a temperature at which specific cleaving occurs; and
c. instructions for performing said cleaving.

31. A kit for reverse transcribing an RNA template with an enzyme, said kit comprising:
a. a container containing a source of magnesium ions;
b. a container containing a source of phosphate ions, wherein said source of magnesium ions and said source of phosphate ions form a precipitate containing magnesium when combined at temperatures below a temperature at which specific reverse transcription occurs; and
c. instructions for performing said reverse transcription.

32. A kit for ligating a nucleic acid sequences with an enzyme, said kit comprising:
a. a container containing a source of magnesium ions;
b. a container containing a source of phosphate ions, wherein said source of magnesium ions and said source of phosphate ions form a precipitate containing magnesium when combined at temperatures below a temperature which specific molecular manipulation occurs; and
c. instructions for performing said ligation.

33. A kit for molecular manipulation of a nucleic acid sequence with an enzyme, said kit comprising:
a. a container containing a reagent comprising a precipitate containing magnesium;
b. instructions for using the precipitate containing magnesium for performing said molecular manipulations.
